(19) United States
(12) Patent Application Publication
(10) Pub. No.: US 2016/0058911 A1
(43) Pub. Date: Mar. 3, 2016

(54) MULTI-STEP METHOD FOR FABRICATING TISSUE ENGINEERING BONE

(71) Applicant: Shanghai Ninth People's Hospital, Shanghai Jiao Tong University School of Medicine, Shanghai (CN)

(72) Inventors: Zhiyong Zhang, Shanghai (CN); Zhenxing Wang, Shanghai (CN); Dingyu Wu, Shanghai (CN); Zhanzhao Zhang, Shanghai (CN)

(21) Appl. No.: 14/935,371

(22) Filed: Nov. 6, 2015

(30) Foreign Application Priority Data
Apr. 1, 2015 (CN) 2015101531816

(51) Int. Cl. A61L 27/38; A61L 27/46; A61L 27/56; A61L 27/36; A61L 27/20

(52) U.S. Cl. CPC A61L 27/3847; A61L 27/365; A61L 27/3834; A61L 27/20; A61L 27/56; A61L 27/46; A61L 2430/02; A61L 2300/64; A61L 2300/412

(57) ABSTRACT

A multi-step method for fabricating a tissue engineering bone comprises the following steps: (1) scaffold repair and pre-vascularization: fabricating a tissue engineering bone scaffold at a bone defect site, and conducting pre-vascularization; and (2) post-implantation of a seed cell for bone tissue engineering: after an inflammatory reaction period, combined with time for micro-vessel in-growth, implanting an osteogenic-induced seed cell into a tissue engineering bone scaffold at the bone defect site. In certain embodiments, the step (2) of post-implantation of a seed cell for bone tissue engineering is performed 7 to 14 days after the step (1) of scaffold repair and pre-vascularization. The method can be used to rapidly fabricate a large section of tissue engineering bone, greatly reduce usage of the seed cell for bone tissue engineering required in repair of per unit volume of bone tissue, and improve the utilization rate of the seed cell.

### TECHNICAL FIELD

The present application relates to the technical field of bone tissue engineering, and in particular, to a multi-step method for fabricating a tissue engineering bone which is used for repair of a bone defect.

### BACKGROUND

Various systemic bone defects caused by wounds, tumors, aging or other factors are a common disease of the surgery system. Clinically, treatment means that are commonly used include autogenous bone grafting, allogeneic bone grafting and using a biological material for repair. However, many problems exist such as damage to the donor bone, potential disease transmission and limited repair effects. The emerging bone tissue engineering technology in recent years is expected to overcome defects of these traditional technologies.

Traditional bone tissue engineering technologies are inoculating a seed cell that is largely expanded in vitro on a three-dimensional degradable biological scaffold having an osteogenicactivity, implanting it into a defect site after in vitro induction for a certain time, and promoting in-growth and absorption of in vivo autogenous vessel as well as osteogenesis of residual bone cells at in-situ bone stump by crawling replacement. When the volume of bone defect is smaller, an effect of complete repair can be achieved generally by stimulation of a seed cell or anosteogenic growth factor. However, for a large section of bone defect, it can hardly achieve a stable and long-term repair effect. It is believed herein that there are five points in respect of the causes, including: 1) rapid death of the seed cell after being implanted: bone marrow mesenchymal stem cells, a common type of seed cell, have been greatly reduced in the first 2 weeks after implantation into a bone defect site, and can hardly be detected in the 4^{th} week, which may be caused by lack of nutrition exchange, low material porosity and the like; 2) slow in-growth of micro-vessels: in general, germination and hyperplasia of a blood capillary at the bone stump needs 1 to 2 weeks, during which a large section of exogenous stem cells implanted into a central area of the tissue engineering bone, have been apoptotic greatly due to insufficient oxygen and lack of nutrition exchange, and could not take a necessary effect of mediation for crawling replacement; and the size factor also determines that the micro-vessel cannot rapidly grow to cover the whole large section of tissue engineering bone; 3) a limited effect of autogenousosteogenic cells in respect of crawling replacement: affected by age and health factors, cell growth ability varies greatly between individuals, and the distances of crawling replacement achieved by osteogenic cells are different, and thus a large section of bone defect can only rely on crawling of osteogenic cells at the bone stump thereof, and can hardly be repaired completely; 4) inflammatory reaction caused by an operation: according to different sites, a strong immunological rejection may generally occur 1 week after an operation, and the inflammatory reaction will be stronger if the volume of the site to be repaired is greater and the wound is larger; and high-dosage antibiotics or anti-rejection drugs injected for anti-infection may also affect repair of a large section of bone defect; and 5) insufficient strength of a scaffold material: common large section of bone defects generally occur in limb bones and lower jawbones, both of which are supporting bone tissues that have high stress requirements and great compressive strength. Common tissue engineering bone materials generally degrade within 1 year, and the time for a large section of new-born bone to reach a bone density consistent with a normal bone varies with age, 2 to 3 years demanded at most. Therefore, a biodegradable material with a slow degradation speed and great compression strength is also urgently demanded.

For the above problems, scholars at home and abroad propose various methods to try solving these problems:

1) A biomaterial that controls releasing of BMP-2 and other growth factorsis used to replace the cell+ material. Such a manner is advantageous in ensuring constant releasing of the osteogenic growth factor, and avoiding effect by cell apoptosis. However, the time of controlled release of BMP-2 is still limited, which cannot fully satisfy requirements for repairing a large section of bone. In addition, dosage is difficultly controlled and a certain potential safety hazards exist. For example, BMP2 products by Medtronic once caused several death accidents in 2008.

2) Vascularization is promoted in manners such as adopting and co-culturing endothelial cells with BMSC, gene transfection of BMSC with VEGF, or pre-embedding of large vessels. It is proved that these manners can promote micro-vessels growing in vivo more rapidly than previously, but the vessel growth rate is still not sufficient.

From the above, due to a large defect volume and different defect sites in a large section of bone defect, adopting the traditional tissue engineering bone technology will obtain different repair effects, and still cannot solve the problem of seed cell apoptosis during a repair process of a large section of bone defect.

### SUMMARY

Embodiments of the present invention provide a multi-step method for fabricating a tissue engineering bone. The method can rapidly fabricate a large section of tissue engineering bone, greatly reduce usage of a seed cell for bone tissue engineering required in repair of per unit volume of bone tissue, and improve the utilization rate of the seed cell.

In order to solve the above technical problem, a multi-step method for fabricating a tissue engineering bone provided by embodiments of the present invention includes the following steps: (1) scaffold repair and pre-vascularization: fabricating a tissue engineering bone scaffold at a bone defect site, and conducting pre-vascularization; and (2) post-implantation of a seed cell for bone tissue engineering: implanting an osteogenic-induced seed cell into a tissue engineering bone scaffold at the bone defect site; where the step (2) of post-implantation of a seed cell for bone tissue engineering is performed 7 to 14 days after the step (1) of scaffold repair and pre-vascularization.

The multi-step method for fabricating a tissue engineering bone provided by the embodiments of the present invention, is divided into 2 steps according to a time sequence: in a first step, fabricating a tissue engineering bone scaffold at a bone defect site, and conducting pre-vascularization; and 7 to 14 days after performing the first step, performing a second step, that is injecting an osteogenic-induced seed cell into a tissue engineering bone scaffold at the bone defect site for implementing osteogenic induction. Compared with the prior art, innovation points of the present invention lie in: (1) changing the idea of implanting a seed cell together with a scaffold material into an in-vivo bone defect site in the traditional tissue engineering bone technology, and adopting theimplanting a scaffoldthat promotes angiogenesisfirst and implanting an osteogenic seed cell at later stage; and (2) for the time interval between the two steps of first repair (i.e. implanting a scaffold that promotes angiogenesis) and post-implantation (i.e. implanting an osteogenic seed cell), the present invention proposes a reasonable scheme that the time interval is 7 to 14 days. According to literature reports and in combination with previous experimental experiences, an acute rejection ends on the 7^{th} day, and vessel germination and hyperplasia occurs within 7 to 14 days. Therefore, the time interval provided by the present invention ensures that the post-implantation occurs at a time point after inflammatory stage and during vessel germination at a bone stump and growth in the scaffold material, thus preventing the seed cell from attacking and rejection by a systemic inflammatory factor during the inflammatory reaction stage, and maximizing survival rate. In general, the repair process of bone defect is two procedures of vessel in-growth and osteogenic cell crawling that are mutually promoted constantly. According to the present invention, pre-fabricating a micro-vessel in vivo with a tissue engineering bone scaffold, and then implanting a seed cell into the scaffold, can avoid the seed cell being killed by an inflammatory reaction at earlier stage of an operation in one aspect, and can also be mutually promoted with the pre-fabricated micro-vessel, thus improving survival rate of the seed cell and ensuring constant osteogenesis. In addition, “post-implantation for osteogenesis” can also achieve crawling replacement at the in-situ bone stump and independent osteogenesis within the scaffold material. The post-implanted seed cell may take part in a crawling replacement effect of autogenous bone cells, and may also form different bone formation islets through ossification in the center of tissue engineering bone far away from the bone stump. The different bone formation islets can achieve mutual connection and promotion, thus ensuring that it is not subject to restriction by the crawling replacement effect of autogenous bone cells, and the whole size of bone defect can be repaired.

In the multi-step method for fabricating a tissue engineering bone provided by the embodiments of the present invention, a method of scaffold repair and pre-vascularization in step (1) may be either of the following two manners: a) implanting a tissue engineering bone scaffold material into a bone defect site, and then implanting a non-induced mesenchymal stem cell; or b) combining a material having an angiogenic activity with a tissue engineering bone scaffold material in vitro, and then implanting the combination into a bone defect site. The material having the angiogenicability may be a non-induced mesenchymal stem cell, or an angiogenic growth factor such as VEGF that can replace a mesenchymal stem cell to promote angiogenesis. Further, the non-induced mesenchymal stem cell is combined with a tissue engineering bone scaffold material in vitro by a method of: first combining a non-induced mesenchymal stem cell with an alginate gel, and then cross-linking the combination with a tissue engineering bone scaffold material. Specifically, the first combining a non-induced mesenchymal stem cell with an alginate gel, and then cross-linking the combination with a tissue engineering bone scaffold material employs a method of: adding double distilled water into an alginate powder, to prepare an alginate gel solution with a mass percentage of 1% to 3%; then mixing the alginate gel solution with a non-induced mesenchymal stem cell precipitate evenly, and adding the mixed solution into a tissue engineering bone scaffold for mixing; finally cross-linking the mixture with 50 to 200 mM calcium chloride for 1 to 5 min. Alginate has wide sources as a natural material, and the safety of in vivo implantation and the stability of effects thereof are both better. In the method of first combining a non-induced mesenchymal stem cell with an alginate gel, and then implanting the combination into a tissue engineering bone scaffold, the alginate gel acts as a cell scaffold material, and takes an effect of wrapping a seed cell.

In the multi-step method for fabricating a tissue engineering bone provided by the embodiments of the present invention, the tissue engineering bone scaffold material used in step (1) may be any tissue engineering bone scaffold material that has osteogenic ability, for example, preferably polycaprolactone, demineralized bone matrix, hydroxyapatite or BETA-tricalcium phosphate. Further, when polycaprolactone is selected as a tissue engineering bone scaffold material of the present invention, the polycaprolactone scaffold material can be prepared by a 3D printing method. The material assumes an alveolate stereoscopic structure and forms a pore inside with a diameter of 0.1 to 0.5 mm. Common large section of bone defects generally occur in sites such as limb bones and lower jawbones, where the bone tissues are supporting bone tissues having high stress requirements and great compressive resistance. In addition, the time for a large section of new-born bone to reach a bone density consistent with a normal bone varies with age, 2 to 3 years demanded at most. The polycaprolactone of an alveolate stereoscopic structure prepared by the above 3D printing technology, is a type of biodegradable material that has a slow degradation speed, great compressive resistance and high mechanical strength. Therefore, it is especially suitable for application as a tissue engineering bone scaffold material in the present invention.

Preferably, in the multi-step method for fabricating a tissue engineering bone provided by the embodiments of the present invention, the implanting an osteogenic-induced seed cell into a tissue engineering bone scaffold at the bone defect site in step (2) employs a method of: under induction by X-rays, injecting an osteogenic-induced seed cell respectively at three positions before, in and behind a bone defect site. Injecting an osteogenic-induced seed cell at multiple positions around a bone defect site, can ensure that the seed cell can be evenly and comprehensively distributed within the tissue engineering bone scaffold at the bone defect site. Particularly, for cases of a large section of bone defect, it can ensure rapid achievement of the repair effect and improve utilization rate of the seed cell.

Preferably, in the multi-step tissue engineering bone provided by the embodiments of the present invention, the seed cell is a mesenchymal stem cell, for example, a bone marrow mesenchymal stem cell, an adipose mesenchymal stem cell, an osteoblast, or the like. Among various seed cells, the allogeneic bone marrow mesenchymal stem cell is preferably selected. The type of cell has been proved as a bone-derived mesenchymal stem cell having relatively best osteogenic ability, and can be used as a source of a tissue engineering bone seed cell for repair of an in-situ bone defect. In combination with an existing bone marrow seed cell bank, adopting a same batch of seed cells can much more assure treatment effects and sources in future clinical application, avoid effects by age and health factors of a patient, and conform to ethical demands.

Finally, in the multi-step tissue engineering bone provided by the embodiments of the present invention, before performing step (1) of scaffold repair and pre-vascularization, the bone defect site is firstly wrapped with a polycaprolactone composite film. Preferably, the polycaprolactone composite film used for wrapping the bone defect site has a thickness of 0.5 to 2 mm, and assumes a planar network structure. The planar network structure has a mesh diameter of 100 to 300 microns. Further, an embodiment of the present invention also provides a method for preparing the polycaprolactone composite film, that is, mixing polycaprolactone with a composite additive respectively according to a mass percentage of 80-100% and 0-20%, and then adopting a 3D printing method to prepare a polycaprolactone composite film. The composite additive in the above preparation method may be BETA-tricalcium phosphate, hydroxyapatite, or coral. The effects of using a polycaprolactone composite film for wrapping a bone defect site include that: in one aspect, the polycaprolactone composite film can take an effect of wrapping the bone defect site, such that after replanting an osteogenic-induced seed cell into the bone defect site, loss of the stem cell caused by flowing of an injected carrier is reduced, and utilization rate of the seed cell is improved; in another aspect, the polycaprolactone composite film has a great strength, thus inhibiting in-growth of surrounding fibrous tissues, reducing possibility of bone un-union caused by space-occupying of the fibrous tissues, and achieving an effect of inducting osteanagenesis; in a further aspect, the polycaprolactone composite film belongs to an inert material, and will not lead to inflammatory reaction after contacting the bone defect site. Additionally, it does not develop under irradiation by X-rays, and will not affect observation and comparison of experimental effects; and the wrapping by the film takes an internal fixation effect similar to a bone lamella, thus maintaining anatomical reduction for a non-weight-bearing bone.

### BRIEF DESCRIPTION OF THE DRAWINGS

FIG. 1 is a flowchart of a multi-step method for fabricating a tissue engineering bone according to certain embodiments of the present invention, where:
1 denotes implanting a tissue engineering bone scaffold at a bone defect site and conducting pre-vascularization; 2 denotes that the bone defect site is in a inflammatory reaction period; 3 denotes implanting a osteogenic-induced seed cell into the bone defect site; and 4 denotes that obvious osteogenesis occurs in the bone defect site;

FIG. 2 is an image of imageological examination that is performed when implanting an osteogenic-induced seed cell into a bone defect site according to an embodiment;

FIG. 3 is an image of imageological examination that is performed 1 month after implanting anosteogenic-induced seed cell into a bone defect site according to an embodiment;

FIG. 4 is an image of imageological examination that is performed 3 months after implanting anosteogenic-induced seed cell into a bone defect site according to an embodiment; and

FIG. 5 is a comparison diagram of imageological examination results for a bone defect site of various experimental groups according to another embodiment.

### DETAILED DESCRIPTION OF THE EMBODIMENTS

To make the objective, technical solutions and advantages of the present invention more clear, the following will describe various implementation manners of the present invention in detail with reference to accompanying drawings. However, a person of ordinary skill in the art may understand that, in various implementation manners of the present invention, many technical details are proposed in order to make this application better understood. Nonetheless, even if without these technical details and various changes and modifications based on the following implementation manners, the technical solutions protected as required by all claims of this application can also be achieved.

### Example 1

### I Building an Animal Model

A rabbit radial bone model has been reported more often as an in-situ bone defect model in various literatures. According to a report, 1.5 cm defect is a critical defect and a cost in animals is economical. As a result, a white adult male rabbit that is 5 months old, and has a 1.5 cm radial bone defect is selected as an animal model of this experiment.

Experimental Grouping

A post-implantation group: specific experimental steps are described as below:

A blank control group: providing no treatment at initial stage, and only injecting 0.3 ml PBS on the 14^{th} day (neither implanting a pre-vascularized seed cell nor implanting an osteogenic seed cell);

Control group 1: adopting a traditional tissue engineering manner, that is, implanting 6 million osteogenic-induced bone marrow mesenchymal stem cells; and

Control group 2: not implanting a pre-vascularized seed cell at initial stage, and only implanting 6 million osteogenic-induced bone marrow mesenchymal stem cells on the 14^{th} day.

(Specific experimental steps of the above blank control group, and the control groups 1 and 2 are omitted)

Specific Experimental Steps of a Post-Implantation Group:

1. Firstly preparing a polycaprolactone (PCL) scaffold material:

(1) taking 15 g polycaprolactoneraw material as a raw material of 3D printing;

(2) first building a geometric model through computer modeling software: designed as an alveolate stereoscopic structure present inside and a pore formed inside with a diameter of 0.5 mm, to finally generate a STL format file; and

(3) printing an entity model: inputting the above STL format file obtained into a 3D printer; setting up printing parameters as demanded, including a nozzle travel path, melting temperature and extrusion speed, and the like; and then printing a three-dimensional model, thus obtaining a polycaprolactone scaffold material that assumes a alveolate stereoscopic structure and forms a pore inside with a diameter of 0.5 mm.

2. Scaffold repair and pre-vascularization:

After mixing 3 million non-induced bone marrow mesenchymal stem cells (BMSC) with 0.5 ml alginate solution with a mass concentration of 1.5%, adding the mixed solution into a 1.5 cm*0.3 cm*0.5 m PCL scaffold material of a alveolate stereoscopic structure that is prepared in the previous step, and then soaking them with 100 mM calcium chloride aqueous solution for cross-linking over 2 min to form a gel, then implanting the gel into a bone defect site.

3. Post-implantation of a seed cell for bone tissue engineering:

Until resolution of an inflammatory reaction after 14 days, during vessel in-growth, a BMSC+pbs aqueous solution that has been angiogenic induced for 7 to 14 days is respectively injected before, in and behind the bone defect site for three times, i.e. prior to, during and after defect cut-off

4. Experimental Results:

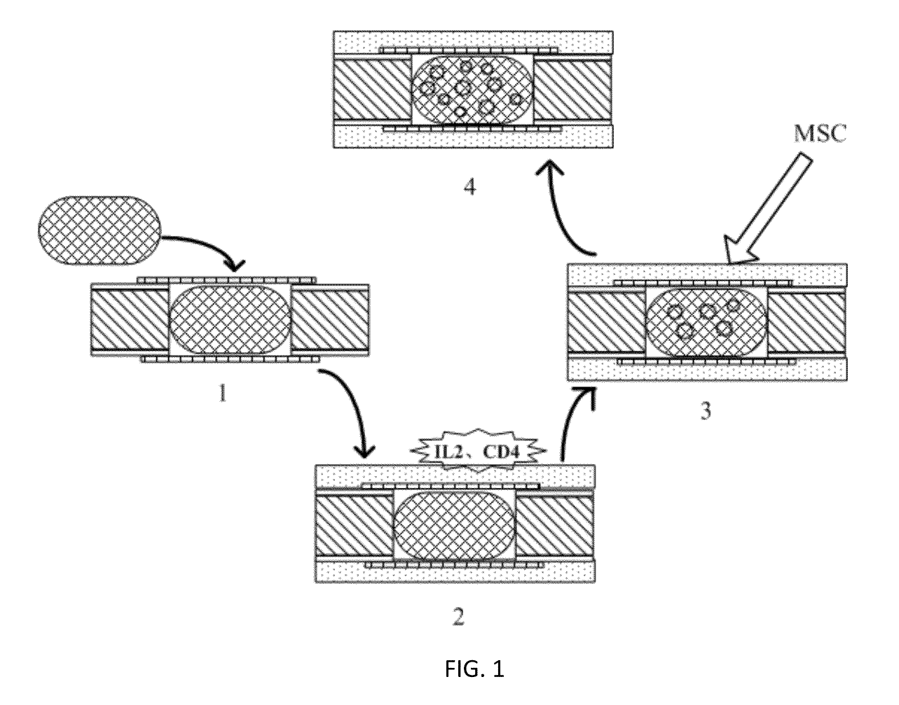

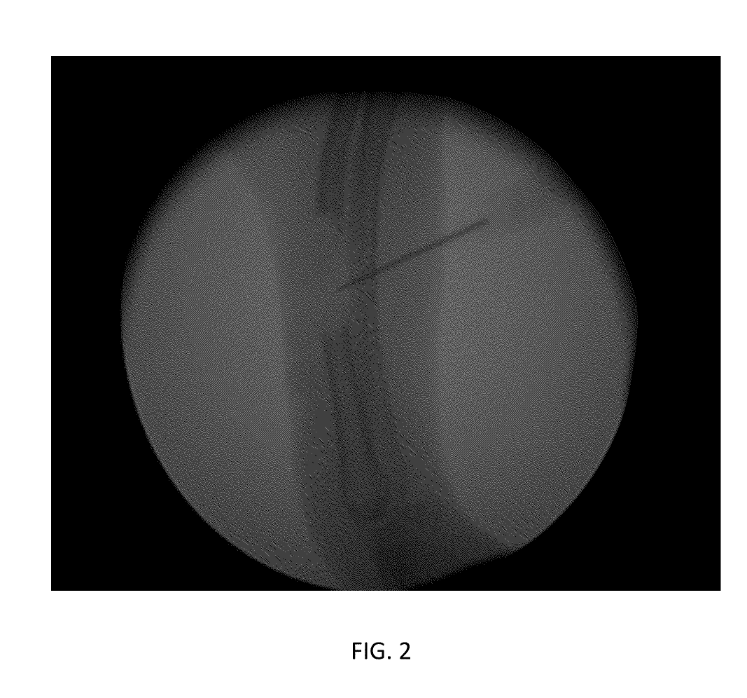

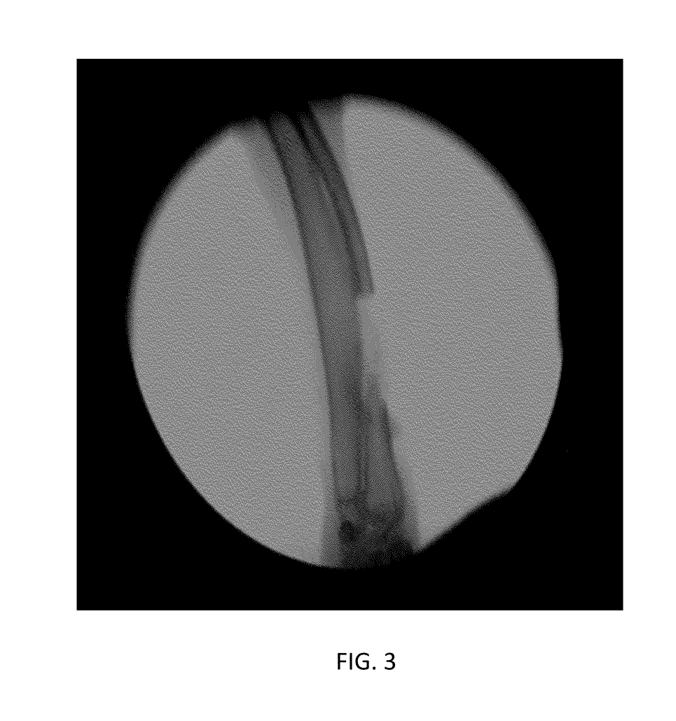

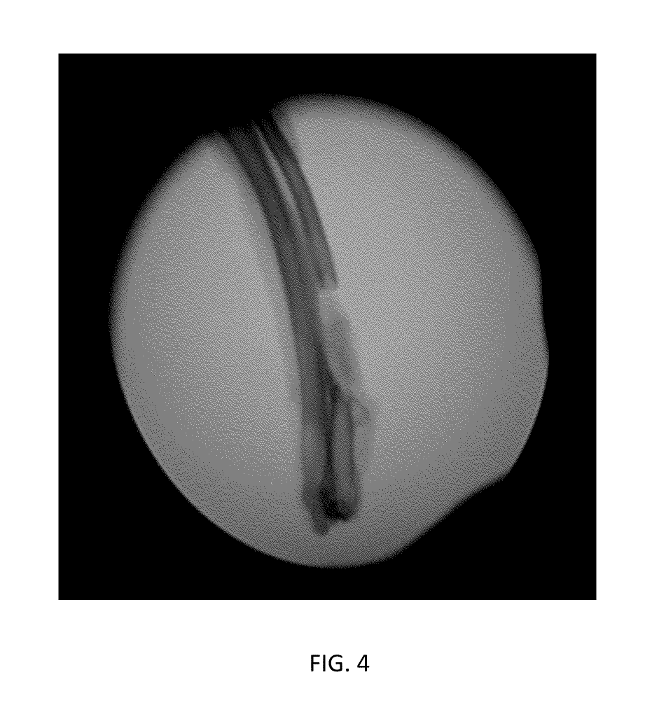

FIGS. 2, 3 and 4 respectively show results of imageological examinations for the post-implantation group that are performed during implantation of an angiogenic induced seed cell into a scaffold, 1 month after the implantation and 3 months after the implantation.

In the post-implantation group, an osteogenesis image emerges 1 month after the implantation of an angiogenic induced seed cell into a scaffold, and distributes along a replantation material with even osteogenesis, while no osteogenesis signs appear in the control groups. In the post-implantation group, obvious osteogenesis occurs 3 months after the implantation of an angiogenic induced seed cell into a scaffold, and a uniform bone tissue is formed along the material at the injection site. The bone tissue has grown into the material, and completely replaced the bone stump and injection site by crawling.

### Example 2

1. Preparing a polycaprolactone (PCL) composite film:

(1) taking 15 g polycaprolactone raw material as a 3D printing material;

(2) first building a geometric model through computer modeling software: designed as a planar network structure with a pore having a diameter of 200 micros and a thickness of 2 mm, and finally generating a STL format file; and

(3) Printing an entity model: inputting the above obtained STL format file into a 3D printer, setting up printing parameters as demanded, including a height of nozzle above a plate, nozzle extrusion speed and nozzle travel path, and the like, and then printing a three-dimensional model, thus obtaining a polycaprolactone composite film that has a thickness of 2 mm and a pore diameter of 200 micros.

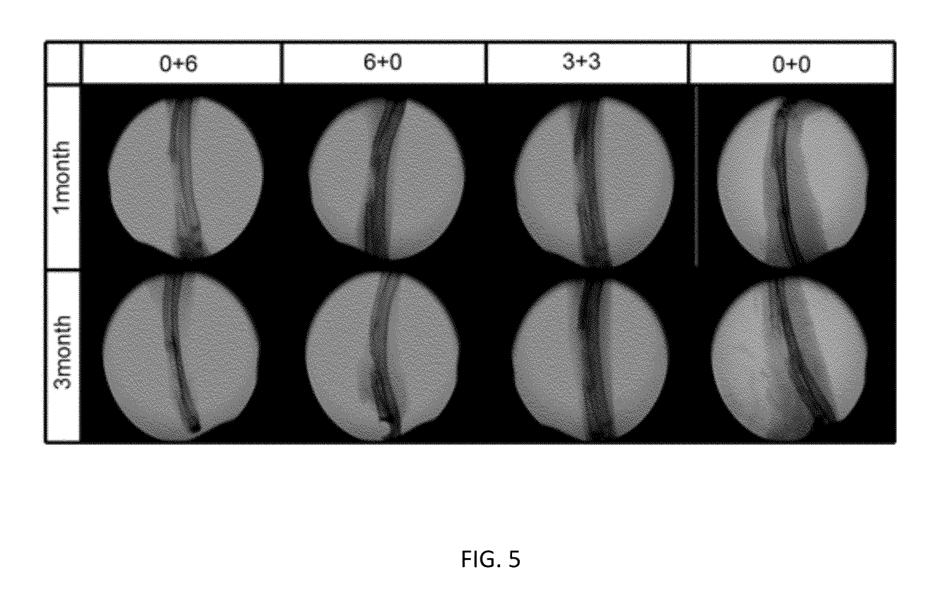

2. Using the polycaprolactone composite film prepared in the previous step to wrap the bone defect site, and then performing multi-step tissue engineering bone fabrication according to experimental design and steps in Example 1. For each experimental group, an imageological examination is performed respectively after 1 month and 3 months, and the effects are compared, as shown in FIG. 5. 0+0 represents the blank control group; 3+3 represents the post-implantation group of the present invention; 6+0 represents the control group 1, i.e. a traditional tissue engineering manner; and 0+6 represents the control group 2. It can be seen from FIG. 5 that, the group 3+3 (i.e. the post-implantation group of the present invention) has the most even osteogenesis and relatively better effects. Moreover, the rabbit radical bones that are wrapped with a PCL composite film in the experimental groups all maintain stability of anatomical structures, where fracture rarely occurs and compared to not wrapping with a PCL composite film, the success rate of osteogenesis is significantly improved.

A person of ordinary skill in the art may understand that, the aforementioned implementation manners are specific embodiments of the present invention, but in practical applications, any change can be made in respect of forms and details, but not beyond the spirit and scope of the present invention.

What is claimed is:

1. A multi-step method for fabricating a tissue engineering bone, comprising:
(1) scaffold repair and pre-vascularization: fabricating a tissue engineering bone scaffold at a bone defect site, and conducting pre-vascularization; and
(2) post-implantation of a seed cell for bone tissue engineering: implanting an osteogenic-induced seed cell into a tissue engineering bone scaffold at the bone defect site;
wherein the step (2) of post-implantation of a seed cell for bone tissue engineering is performed 7 to 14 days after the step (1) of scaffold repair and pre-vascularization.

2. The multi-step method for fabricating a tissue engineering bone according to claim 1, wherein the step (1) of scaffold repair and pre-vascularization employs a method of:
a) implanting a tissue engineering bone scaffold material into a bone defect site, and then implanting a non-induced mesenchymal stem cell; or
b) combining a material having an angiogenic activity with a tissue engineering bone scaffold material in vitro, and then implanting the combination into a bone defect site.

3. The multi-step method for fabricating a tissue engineering bone according to claim 2, wherein the material having the angiogenic activity is a non-induced mesenchymal stem cell or an angiogenic growth factor.

4. The multi-step method for fabricating a tissue engineering bone according to claim 3, wherein the non-induced mesenchymal stem cell is combined with a tissue engineering bone scaffold material in vitro by a method of: first combining a non-induced mesenchymal stem cell with an alginate gel, and then cross-linking the combination with a tissue engineering bone scaffold material.

5. The multi-step method for fabricating a tissue engineering bone according to claim 4, wherein the first combining a non-induced mesenchymal stem cell with an alginate gel, and then cross-linking the combination with a tissue engineering bone scaffold material employs a method of: adding double distilled water into an alginate powder, to prepare an alginate gel solution with a mass percentage of 1% to 3%; then mixing the alginate gel solution with a non-induced mesenchymal stem cell precipitate evenly, and adding the mixed solution into a tissue engineering bone scaffold for mixing; finally cross-linking the mixture with 50 to 200 mM calcium chloride for 1 to 5 min.

6. The multi-step method for fabricating a tissue engineering bone according to claim 1, wherein the tissue engineering bone scaffold material in the step (1) is polycaprolactone, demineralized bone matrix, hydroxyapatite or BETA-tricalcium phosphate.

7. The multi-step method for fabricating a tissue engineering bone according to claim 6, wherein the polycaprolactone scaffold material is prepared by a 3D printing method, assumes an alveolate stereoscopic structure and forms a pore inside with a diameter of 0.1 to 0.5 mm.

8. The multi-step method for fabricating a tissue engineering bone according to claim 1, wherein the implanting an osteogenic-induced seed cell into a tissue engineering bone scaffold at the bone defect site in the step (2) employs a method of: under induction by X-rays, injecting an osteogenic-induced seed cell respectively at three positions before, in and behind a bone defect site.

9. The multi-step method for fabricating a tissue engineering bone according to claim 1, wherein the seed cell is a mesenchymal stem.

10. The multi-step method for fabricating a tissue engineering bone according to claim 9, wherein the mesenchymal stem is a bone marrow mesenchymal stem cell, an adipose mesenchymal stem cell or an osteoblast.

11. The multi-step method for fabricating a tissue engineering bone according to claim 10, wherein before performing the step (1), a bone defect site is wrapped with a polycaprolactone composite film.

12. The multi-step method for fabricating a tissue engineering bone according to claim 11, wherein the polycaprolactone composite film has a thickness of 0.5 to 2 mm and assumes a planar network structure, and the planar network structure has a mesh diameter of 100 to 300 microns.

13. The multi-step method for fabricating a tissue engineering bone according to claim 11, wherein the polycaprolactone composite film is prepared by a method of: mixing polycaprolactone with a composite additive respectively according to a mass percentage of 80-100% and 0-20%, and then adopting a 3D printing method to prepare the polycaprolactone composite film.

14. The multi-step method for fabricating a tissue engineering bone according to claim 13, wherein the composite additive is BETA-tricalcium phosphate, hydroxyapatite, or coral.
